(19) United States
(12) Patent Application Publication
(10) Pub. No.: US 2006/0025761 A1
(43) Pub. Date: Feb. 2, 2006

(54) Linear-array radio frequency resections

(76) Inventors: Lee B. Riley, Bethlehem, PA (US); Mehran Habibi, Bethlehem, PA (US)

Correspondence Address: BLANK ROME LLP, 600 NEW HAMPSHIRE AVENUE, N.W., WASHINGTON, DC, 20037, US

(21) Appl. No.: 10/901,134

(22) Filed: Jul. 29, 2004

(51) Int. Cl. A61B 18/14

(52) U.S. Cl. 606/41

(57) ABSTRACT

An apparatus and method for ablation of a resection plane in an organ of the body, is disclosed. A linear-array probe with a plurality of tines penetrates a cross-section of the organ. A radio frequency generator is connected to the linear-array probe via wire conductors. The radio frequency generator generates a current that produces radio frequency thermal energy at the tines to ablate a plane of the cross-section of the organ along a line of the tines. Size of the ablated plane depends on the plurality of tines in the linear-array probe. The resection plane is dependent on alignment of the tines in the linear-array probe.

### FIELD OF THE INVENTION

The present invention is generally related to surgical procedures on the human body and, more particularly, is related to a method and apparatus for ablation of a resection plane in an organ of the body using radio frequency technology.

### BACKGROUND OF THE INVENTION

Because of their vascularity, solid organs pose particular problems concerning blood loss during surgery which needs to be approached differently from blood loss problems when carrying out other types of surgery. One of the most important solid organs in this context is the liver.

Thus, blood loss, bile leak and postoperative liver function are the main concerns for surgeons operating on the liver, surgical resection remaining the only potentially curative procedure for dealing with hepatic tumors. Increased intraoperative bleeding is associated with postoperative complications and shorter long-term survival. In addition, it is a major parameter in evaluating results of liver resection since it affects postoperative morbidity, mortality and long term survival in malignant disease. Operative blood loss can occur during dissection, parenchymal transection and revascularization.

Different techniques have been developed to allow safe liver resection. Surgeons can decrease intraoperative blood loss by limiting or occluding the inflow occlusion, performing a careful and oftentimes time-consuming parenchymal dissection, or both. Inflow occlusion can be obtained by means of Pringle maneuver or total vascular exclusion. Parenchymal division can be performed using a scalpel, crushing the tissue with the finger or clamps, using ultrasonic dissectors and hydrodissectors or stapling devices. Vascular and biliary structures larger than 2 mm require ligation and division. However, late bleeding and bile leak are possible and may be caused by insufficient ligation or oozing from the resection surface secondary to tearing of small vessels.

Radio frequency thermal energy has increasingly been used to locally ablate unresectable hepatic disease. Electricity converts current into thermal energy by ionic agitation and in so doing causes proteins to denature resulting in coagulative necrosis.

The spleen and kidneys are other vascular organs in the body with the potential for excessive bleeding if traumatized. For example, when the spleen starts to bleed because of an accident or iatrogenic trauma during surgery in adults, a splenectomy is usually performed to arrest bleeding and to save life.

Tumors of parenchymatous organs or tumors penetrating hollow internal organs are known to be treatable by hyperthermia induced by radio frequency. Such a treatment is made with suitable radio frequency generators provided with a passive electrode to be applied outside the patient's body, and with an active electrode to be inserted into the patient's body and directly contacting the tumor. The known active electrodes consist of variously sized needles which are inserted into the patient's body by passing them through several layers of the body tissue. This results in drawbacks due to passing through tissues other than the one to be treated, some of which are not easily pierced by large-sized catheters.

Open surgical resection of a large sector of the liver is done routinely to remove regions where cancerous tumors are believed to exist. Such a procedure is possible only after using imaging techniques to determine the exact locations where the cancerous tumors are believed to exist. Such operations are technically challenging, morbid, and dangerous, often resulting in fatalities. They require an expensive and time-consuming surgical procedure. For a person in frail health or with significant health problems, undergoing such major surgery can be prohibitive or lead to extended recovery periods, which are inconvenient and costly.

In view of the above-referenced difficulties, it is understandable that radio frequency probes which simultaneously cut and coagulate are preferred. However, current probes are poorly adapted to certain activities, such as cutting narrow tendon or ligaments. In cases where a resection plane is desired, the plane was established by overlapping multiple sphere-shaped ablations using, for example, a standard spherical 3.5 cm probe. This method is impractical and very time-consuming.

Thus, a need exists in the art for a method of ablating large volumes of tissue having cancerous areas, minimal blood loss and a shorter surgery time than that currently available with standard spherical probes using radio frequency technology.

### SUMMARY OF THE INVENTION

Embodiments of the present invention provide an apparatus and method for ablation of a resection plane in an organ of the body using a linear-array probe and radio frequency technology.

Briefly described, in architecture, one preferred embodiment of the invention can be implemented as follows. The apparatus includes a linear-array probe with a plurality of tines. The tines are of a predetermined length and are spaced a predetermined distance apart. The tines penetrate a cross-section of the organ which has been exposed by conventional surgical techniques. A radio frequency generator is connected to the tines through the super-structure of the linear-array probe and supplies radio frequency current to the tines. A connection means, such as wire conductors, connect the radio frequency generator to the tines. The thermal energy generated from the tines ablates a plane in the organ, the size of which is dependent on the number of tines deployed from the linear-array probe. A method, employing the above-referenced apparatus, is used to ablate the section plane of the organ.

In another embodiment of the invention, an adapter is used with the linear-array probe. The adapter assists in stabilizing the organ during and after the ablation procedure.

In another embodiment of the invention, the tines in the linear-array probe are structured to individually screw into the organ. Alligator clips may be used to connect the wire conductors to the tines.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a perspective drawing of a preferred embodiment of the invention; and

FIG. 2 is a perspective drawing of an embodiment of the invention with the adapter to stabilize the organ.

### DETAILED DESCRIPTION OF THE PREFERRED EMBODIMENT

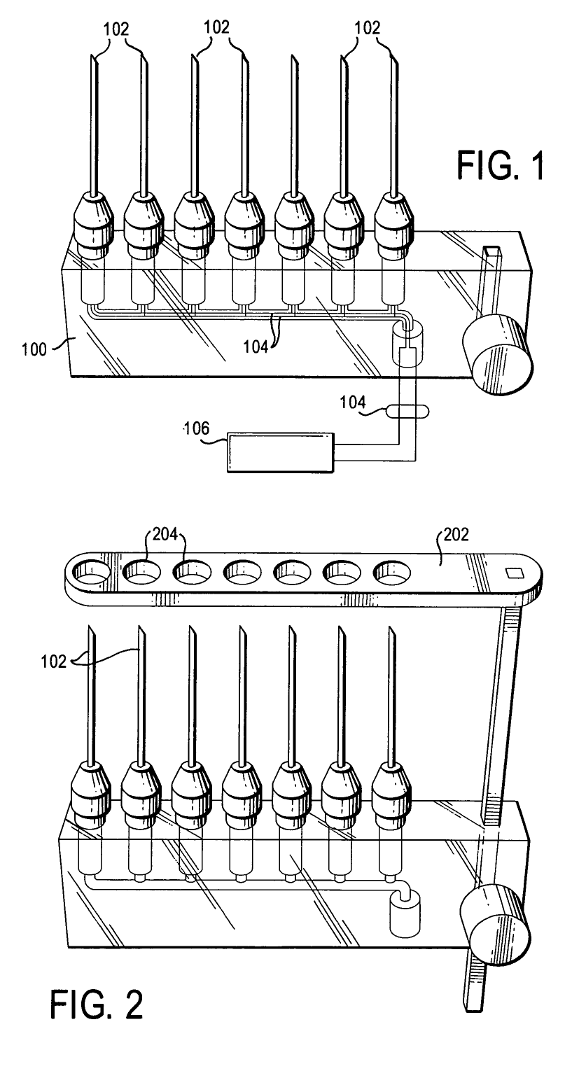

FIG. 1 illustrates a preferred embodiment of a linear-array probe 100. The linear-array probe 100 includes a plurality of tines 102 for piercing and penetrating into a cross-section of an organ of the body. The tines 102 are of a predetermined length with a preferred length sufficient to traverse the cross-section of the organ. The number of tines 102 in the linear-array probe 100 determine the size of a desired ablated plane of the organ. In the preferred embodiment, seven tines are used, each with a length sufficient to traverse the cross section of the liver. The tines 102 are spaced a predetermined distance apart on the linear-array probe. The preferred distance is dependent on the desired ablation line-of-plane needed in the surgery.

The alignment of the tines 102 along the super-structure of the linear-array probe 100 dictates the ablation line-of-plane of the resection section. Since tumors and target resection sections vary in size, the more closely the ablation follows the outline of the target resection, the lesser the loss of tissue, and, minimal blood loss. If the target resection (tumor, resection tissue section) requires a slightly curved ablation, then the tines 102 are aligned to present a curved line-of-plane to accommodate. Thus, the alignment of the tines 102 can be varied to accommodate the desired ablation plane which is governed by the outline of the target resection tissue section.

A radiofrequency generator 106 is provided to generate a current that is converted to thermal energy at the tines 102. The thermal energy ablates the desired plane of the organ. Wire conductors 104 connect the radiofrequency generator 106 to the linear-array probe 100 via the tines 104 FIG. 2 illustrates an embodiment of the linear-array probe 100 including an adapter 202. The adapter 202 is a template with slots 204 to accommodate the tines 102. The adapter 202 stabilizes the organ when the tines 102 are urged into and withdrawn from the organ.

In practice, the organ (not shown) is exposed through standard medical surgical techniques. To transect the organ using the present invention, the linear-array probe 100 is positioned over the organ. The tines 102 are positioned to pierce the organ and are urged through the cross-section of the organ. The linear-array probe 100 is positioned to allow a majority of the tines 102 to pierce and penetrate the cross-section of the organ. The length of the tines 102 is sufficient to allow the tines 102 to penetrate through the cross-section of the organ and come out an opposing end.

Once the linear-array probe is in position, wire conductors 104 connect the linear-array probe 100 to a radio frequency generator 106. The radio frequency generator 106 generates an RF current to the tines 102 which is converted into thermal energy that ablates the organ in a plane along a line of the tines 102. Once resection of the cross-section of the organ is complete, the adapter 202 stabilizes the organ as the tines 102 are withdrawn. Because of the radio frequency ablation along a plane of the cross-section of the organ, the surgical procedure is quicker, practically bloodless and more efficient than available with a standard spherical probe.

In another embodiment of the invention, the linear-array probe incorporates the radio frequency generator within the super-structure, thus obviating the need for the connection means.

It should be emphasized that the above-described embodiments of the present invention, particularly, any preferred embodiments, are merely possible examples of implementations, merely set forth for a clear understanding of the principles of the invention. Many variations and modifications may be made to the above-described embodiments of the invention without departing substantially from the spirit and principles of the invention. All such modifications and variations are intended to be included herein within the scope of this disclosure and the present invention and protected by the following claims.

What is claimed is:

1. An apparatus for ablation of a resection plane in an organ of the body, comprising:
a linear-array probe for penetrating a cross-section of the organ;
a radio frequency wave generator for generating radio frequency waves; and
means for connecting said radio frequency wave generator to said linear-array probe.

2. The apparatus according to claim 1, wherein the linear-array probe includes at least two tines of predetermined length to traverse the cross-section of the organ.

3. The apparatus according to claim 1, wherein the connection means includes wire conductors.

4. The apparatus according to claim 1, wherein the linear-array probe includes at least seven tines of predetermined length.

5. The apparatus according to claim 4, wherein the length of the tines is variable and sufficient to penetrate the cross-section of the organ.

6. An apparatus for ablation of a resection plane in an organ of the body, comprising:
a linear-array probe including a plurality of tines that penetrate a cross-section of the organ;
a radio frequency wave generator connected to the linear-array probe; and
wire conductors that connect the radio frequency wave generator to the linear-array probe.

7. The apparatus according to claim 6, wherein the tines include a variable length sufficient to penetrate the cross-section of the organ.

8. The apparatus according to claim 6, wherein the linear-array probe includes at least seven tines.

9. A method for ablation of a resection plane in an organ of the body, the method comprising:
providing a linear-array probe for penetrating a cross-section of the organ;
piercing the organ with a plurality of tines located on the linear-array probe;
providing a radio frequency wave generator for supplying RF energy to the tines on the linear-array probe;
connecting said radio frequency wave generator to the tines; and
resecting a plane of the cross-section from the organ using the tines.

10. The method of claim 9, further comprising penetrating the organ of the body to a predetermined depth via traversing the cross-section of the organ using the tines.

11. The method of claim 9, comprising penetrating the organ of the body with at least seven tines.

12. The method of claim 9, comprising connecting from the radio frequency wave generator to the tines using wire conductors.

13. A device for ablating a resection plane in an organ of the body, comprising:
a multi-electrode means for simultaneously penetrating a cross-section of the organ;
means for supplying high energy waves in a radio frequency range to the multi-electrode means; and
means for connecting the multi-electrode means to the means for supplying high energy waves.

14. An apparatus for ablation of a resection plane in the liver of the body, comprising:
a linear-array probe for penetrating a cross-section of the liver;
a radio frequency wave generator for generating radio frequency waves; and
means for connecting said radio frequency wave generator to said linear-array probe.

15. The apparatus according to claim 14, wherein the linear-array probe includes at least two tines of predetermined length to traverse the cross-section of the liver.

16. The apparatus according to claim 14, wherein the connection means includes wire conductors.

17. The apparatus according to claim 14, wherein the linear-array probe includes at least seven tines of predetermined length.

18. The apparatus according to claim 17, wherein the length of the tines is variable and sufficient to penetrate the cross-section of the liver.

19. An apparatus for ablation of a resection plane in the liver of the body, comprising:
a linear-array probe including a plurality of tines that penetrate a cross-section of the liver;
a radio frequency wave generator connected to the linear-array probe; and
wire conductors that connect the radio frequency wave generator to the linear-array probe.

20. The apparatus according to claim 19, wherein the tines include a variable length sufficient to penetrate the cross-section of the liver.

21. The apparatus according to claim 19, wherein the linear-array probe includes at least seven tines.

22. A method for ablation of a resection plane in the liver of the body, the method comprising:
providing a linear-array probe for penetrating a cross-section of the liver;
piercing the liver with a plurality of tines located on the linear-array probe;
providing a radio frequency wave generator for supplying RF energy to the tines on the linear-array probe;
connecting said the radio frequency wave generator to the tines; and
resecting a plane of the cross-section from the liver using the tines.

23. The method of claim 22, further comprising penetrating the liver to a predetermined depth via traversing the cross-section of the liver using the tines.

24. The method of claim 22, comprising penetrating the liver with at least seven tines.

25. The method of claim 22, comprising connecting from the radio frequency wave generator to the tines using wire conductors.

26. A device for ablating a resection plane in the liver of the body, comprising:
multi-electrode means for simultaneously penetrating a cross-section of the liver;
means for supplying high energy waves in a radio frequency range to the multi-electrode means; and
means for connecting the multi-electrode means to the means for supplying high energy waves.

27. An apparatus for ablation of a resection plane in a kidney of the body, comprising:
a linear-array probe for penetrating a cross-section of the kidney;
a radio frequency wave generator connected to said linear-array probe for generating radio frequency waves; and
means for connecting said radio frequency wave generator to said linear-array probe.

28. The apparatus according to claim 27, wherein the linear-array probe includes at least two tines of predetermined length to traverse the cross-section of the kidney.

29. The apparatus according to claim 27, wherein the connection medium includes wire conductors.

30. The apparatus according to claim 27, wherein the linear-array probe includes at least seven tines of predetermined length.

31. The apparatus according to claim 30, wherein the length of the tines is variable and sufficient to penetrate the cross-section of the kidney.

32. An apparatus for ablation of a resection plane in a kidney of the body, comprising:
a linear-array probe including a plurality of tines that penetrate a cross-section of the kidney;
a radio frequency wave generator connected to the linear-array probe; and
wire conductors that connect the radio frequency wave generator to the linear-array probe.

33. The apparatus according to claim 32, wherein the tines include a variable length sufficient to penetrate the cross-section of the kidney.

34. The apparatus according to claim 322, wherein the linear-array probe includes at least seven tines.

35. A method for ablation of a resection plane in a kidney of the body, the method comprising:
providing a linear-array probe for penetrating a cross-section of the kidney;
piercing the kidney with a plurality of tines located on the linear-array probe;
providing a radio frequency wave generator for supplying RF energy to the tines on the linear-array probe;
connecting said radio frequency wave generator to the tines; and
resecting a plane of the cross-section from the kidney using the tines.

36. The method of claim 35, further comprising penetrating the kidney to a predetermined depth via traversing the cross-section of the kidney using the tines.

37. The method of claim 35, comprising penetrating the kidney with at least seven tines.

38. The method of claim 35, comprising connecting from the radio frequency wave generator to the tines using wire conductors.

39. A device for ablating a resection plane in a kidney of the body, comprising:
multi-electrode means for simultaneously penetrating a cross-section of the kidney;
means for supplying high energy waves in a radio frequency range to the multi-electrode means; and
means for connecting the multi-electrode means to the means for supplying high energy waves.

40. The apparatus according to claim 6, wherein the linear-array probe includes an adapter to stabilize the organ.

41. The apparatus according to claim 40, wherein the adapter includes a template with a plurality of slots to accommodate the tines when the tines penetrate the cross-section of the organ.
